(19) United States
(12) Patent Application Publication
(10) Pub. No.: US 2010/0185029 A1
(43) Pub. Date: Jul. 22, 2010

(54) TWO STEP PROCESS FOR THE MANUFACTURE OF HYDROFLUOROOLEFINS

(75) Inventors: Maher Y. Elsheikh, Wayne, PA (US); Philippe Bonnet, Lower Merion, PA (US); Laurent Wendlinger, Soucieu en Jarrest (FR)

Correspondence Address: ARKEMA INC.;PATENT DEPARTMENT - 26TH FLOOR, 2000 MARKET STREET, PHILADELPHIA, PA, 19103-3222, US

(73) Assignee: ARKEMA INC., Philadelphia, PA (US)

(21) Appl. No.: 12/664,214

(22) PCT Filed: Jun. 27, 2008
(86) PCT No.: PCT/US2008/068510

§ 371 Date: Dec. 11, 2009

Related U.S. Application Data
(60) Provisional application No. 60/946,407, filed on Jun. 27, 2007.

(51) Int. Cl. C07C 17/23; C07C 17/093

(52) U.S. Cl. 570/157; 570/155; 570/156; 570/158

(57) ABSTRACT

The present invention discloses a process for the synthesis of hydrofluoroolefins (HFO). The process is based on the following two steps: the liquid phase fluorination of a hydrochloropropane(s) to hydrofluoropropane(s) (HFP) followed by the dehydrofluorination of the hydrofluoropropane(s) (HFP) to hydrofluoroolefins (HFO).

### FIELD OF THE INVENTION

The present invention relates to a process for the manufacture of hydrofluoroolefins. More particularly, the present invention relates to a process for manufacturing hydrofluoropropene in a two step process. The two step process comprises fluorination of a hydrochloropropane to hydrfluoropropane, followed by dehydrofluorination of the latter compound to form hydrofluoropropene.

### BACKGROUND OF THE INVENTION

The Montreal Protocol for the protection of the ozone layer, mandate the phase out of the use of chlorofluorocarbons (CFCs). Materials more “friendly” to the ozone layer such as hydrofluorocarbon (HFC) eg 134a replaced chlorofluorocarbons. The latter compounds have proven to be green house gases, causing global warming and were regulated by the Kyoto Protocol on Climate Change. The emerging replacement materials, hydrofluoropropenes, were shown to be environmentally acceptable i.e. have zero ozone depletion potential (ODP) and acceptable low GWP. The present invention describes a process for manufacturing of hydrofluoropropenes.

### DESCRIPTION OF THE INVENTION

The present invention provides a process for producing a hydrofluoroolefin such a hydrofluoropropene of the formula C₃H₍ₐ₊ₓ₋₁₎F₇₋₍ₐ₊ₓ₎ where a=0,1, 2, 3 or 4 and x=0, 1 and a+x is one or more, comprising the steps of:

a) fluorinating a hydrochloropropane with HF to form a hydrofluoropropane of the formula C₃Hₐ₊ₓF₈₋₍ₐ₊ₓ₎; and

b) dehydrofluorinating the hydrofluoropropane to form hydrofluoropropene of the formula C₃H₍ₐ₊ₓ₋₁₎F₇₋₍ₐ₊ₓ₎.

The first step of the present process comprises the catalyzed liquid phase fluorination of hydrochloropropane to hydrofluoropropane. The hydrochloropropane starting material maybe be prepared by any preparation process know in the art. For example, the formation of hydrochlorocarbon via telomerization of a single carbon group having from one to four chlorine substitutions such as CH₃Cl (methylchloride) or CCl₄ (carbon tetrachloride) telomerized with an olefin such as CCl₂═CCl₂ (perchloroethylene) or CH₂═CCl₂ (dichloroethylene) in the presence of an appropriate catalyst.

The first step of the present invention, fluorination of a hydrochloropropane (HCP) to form a hydrofluoropropane (HFP) proceeds via Formula 1.

C₃Hₐ₊ₓCl₈₋₍ₐ₊ₓ₎+8-(a+x)HF→C₃Hₐ₊ₓF₈₋₍ₐ₊ₓ₎+8-(a+x)HCl Formula 1

Representative reactions are shown in Table 1:

TABLE 1

| C₃Hₐ₊ₓCl₈₋₍ₐ₊ₓ₎ | | Example | C₃Hₐ₊ₓF₈₋₍ₐ₊ₓ₎ | Example | |
|---|---|---|---|---|---|
| | | HCP¹ reactant | | HFP² product | |
| a + x = 3 | C₃H₃Cl₅ | CCl₃CH₂CHCl₂ | C₃H₃F₅ | CF₃CH₂CHF₂ | 245fa |
| | | CCl₃CCl₂CH₃ | C₃H₃F₅ | CF₃CF₂CH₃ | 245cb |
| a + x = 4 | C₃H₄Cl₄ | CCl₃CH₂CH₂Cl | C₃H₄F₄ | CF₃CF₂CH₃ | 254fb |

| | | | |
|---|---|---|---|
| C₃Hₐ₊ₓCl₈₋₍ₐ₊ₓ₎ | Example | C₃Hₐ₊ₓF₈₋₍ₐ₊ₓ) | Example |
| | HCP¹ reactant | | HFP² product |

¹HCP is hydrochlorocarbon
²HFP is hydrofluoropropane

The first step is a liquid fluorination process using HF as a fluorinating agent and proceeds through Cl—F exchanges. A liquid phase fluorination process present advantages versus a gas phase fluorination process as it leads to a better selectivity towards the desired product with a much lower required energy level. However, for C2 and C3 compounds, liquid phase fluorination usually achieves partial Cl exchange and often a second step to achieve full Cl replacement is required. The present inventors found that a full replacement of Cl of the starting hydrochloropropane is achievable in a liquid phase process in the presence of a catalyst which is advantageously chosen from the derivatives of metals of groups 3, 4, 5, 13, 14 and 15 of the Periodic Table of the elements (IUPAC 1988) and their mixtures (groups of the Periodic Table of the elements which were previously called IIIA, IVa, IVb, Va, Vb and VIb). The derivatives of the metals are intended to mean the hydroxides, oxides and the organic or inorganic salts of these metals, as well as their mixtures. Those particularly adopted are the titanium, tantalum, molybdenum, boron, tin and antimony derivatives. The catalyst is preferably chosen from the derivatives of metals of groups 14 (IVa) and 15 (Va) of the Periodic Table of the elements, and more particularly from tin and antimony derivatives. In the process according to the invention the preferred derivatives of the metals are the salts and these are preferably chosen from the halides and more particularly from chlorides, fluorides and chlorofluorides. Particularly preferred fluorination catalysts according to the present invention are tin and antimony chlorides, fluorides and chlorofluorides, especially tin tetrachloride and antimony pentachloride. Antimony pentachloride is very particularly recommended. In addition to the above mentioned Lewis acids catalyst, an ionic liquid derived from antimony, titanium, niobium and tantalum is suitable for liquid phase fluorination processes. A description of the preparation of these catalysts is disclosed in the U.S. Pat. No. 6,881,698 incorporated herein by reference.

In the case where the catalyst is selected from metal fluorides and chlorofluorides, these can be obtained from a chloride which is subjected to an at least partial fluorination. This fluorination may, for example, be carried out by means of hydrogen fluoride, before the catalyst is brought into contact with the hydrochloropropane. In an alternative form, it may be carried out in situ, during the reaction of the hydrochloropropane with hydrogen fluoride.

The quantity of catalyst used can vary within wide limits. It is generally at least 0.001 mole of catalyst per mole of the hydrochlorocarbon. It is preferably at least 0.01 mole of catalyst per mole of the hydrochloropropane. In principle there is no upper limit to the quantity of catalyst used. For example, in a process carried out continuously in liquid phase, the molar ratio of the catalyst to the hydrochloropropane may reach 1000. In practice, however, at most approximately 5 moles of catalyst are generally employed per mole of the hydrochloropropane. Approximately 1 mole is preferably not exceeded. In a particularly preferred manner, approximately 0.5 moles of catalyst per mole of the hydrochloropropane are generally not exceeded.

The molar ratio of hydrogen fluoride to the hydrochloropropane used is generally at least 5. The work is preferably done with a molar ratio of at least 8. The molar ratio of hydrogen fluoride to the hydrochloropropane used generally should not exceed 100 and preferably does not exceed 50.

The temperature at which the hydrofluorination is performed is generally at least 50° C. and is preferably at least 80° C. The temperature generally should not exceed 150° C. and preferably does not exceed 130° C. With antimony pentachloride as the catalyst, good results are obtained at a temperature of about 100 to 120° C. The catalyst is activated with HF in such away that the activated catalyst has the general formula SbFₓCl_{y}, x+y=5. The activation process is carried out prior to admitting the reactants to the reaction vessel.

The first step of the process according to the present invention is carried out in liquid phase. The pressure is chosen so as to keep the reaction mixture in liquid form. The pressure used varies as a function of the temperature of the reaction mixture. It is generally from 2 bar to 40 bar. The work is preferably carried out at a temperature and pressure at which, furthermore, the hydrofluoropropane produced is at least partially in gaseous form, which enables it to be easily isolated from the reaction mixture.

The process according to the invention may be carried out continuously or batch wise. It is to be understood that, in a noncontinuous process, the quantity of catalyst used is expressed in relation to the initial quantity of the hydrochloropropane used and, in a continuous process, in relation to the stationary quantity of the hydrochloropropane present in the liquid phase.

The residence time of the reactants in the reactor must be sufficient for the reaction of the hydrochloropropane with hydrogen fluoride to take place with an acceptable yield. It can easily be determined as a function of the operating conditions adopted.

The processes according to the invention must be carried out in a corrosion resistant reactor, such as one coated with a fluorpolymer resistant to both HF and the catalyst. It is advantageous to separate the hydrofluoropropane and the hydrogen chloride from the reaction mixture as they are being formed and to keep in, or return to, the reactor the unconverted reactants, as well as any hydrochlorofluoropropanes possibly formed by incomplete fluorination of the feed stock hydrochloropropane. To this end the process according to the invention is advantageously carried out in a reactor equipped with a device for drawing off a gas stream, this device consisting, for example, of a distillation column and a reflux condenser mounted above the reactor. By means of suitable control, this device makes it possible to draw off in vapor phase the hydrofluoropropane and hydrogen chloride which are produced while keeping in the reactor, in the liquid state, the unconverted hydrochloropropane and most of the hydrogen fluoride, as well as, where appropriate, most of the co-products resulting from partial fluorination of the hydrochloropropane.

The second step, dehydrofluorination of the hydrofluoropropane produced in the first step is carried out in the vapor phase. Vapor phase dehydrofluorination of the hydrofluoropropane may be carried out using typical dehydrofluorination catalysts. Generally, the present dehydrofluorination may be carried out using any dehydrofluorination catalyst known in the art. These catalysts include, but are not limited to, aluminum fluoride; fluorided alumina; metals on aluminum fluoride; metals on fluorided alumina; oxides, fluorides, and oxyfluorides of magnesium, zinc and mixtures of magnesium and zinc and/or aluminum; lanthanum oxide and fluorided lanthanum oxide; chromium oxides, fluorided chromium oxides, and cubic chromium trifluoride; carbon, acid-washed carbon, activated carbon, three dimensional matrix carbonaceous materials; and metal compounds supported on carbon. The metal compounds are oxides, fluorides, and oxyfluorides of at least one metal selected from the group consisting of sodium, potassium, rubidium, cesium, yttrium, lanthanum, cerium, praseodymium, neodymium, samarium, chromium, iron, cobalt, rhodium, nickel, copper, zinc, and mixtures thereof.

The second step of the present invention, the dehydrofluorination of the hydrofluoropropane to form a hydrofluoroolefine HFO proceeds via Formula 2

C₃Hₐ₊ₓF₈₋₍ₐ₊ₓ₎→C₃H₍ₐ₊ₓ₋₁₎F₇₋₍ₐ₊ₓ₎+HF Formula 2

Representative reactions are shown in Table 2:

TABLE 2

| Starting material | | Example | | Product | Example HFO | |
|---|---|---|---|---|---|---|
| HFPC₃Hₐ₊ₓF₈₋₍ₐ₊ₓ₎ | | HFP | | C₃H₍ₐ₊ₓ₋₁₎F₇₋₍ₐ₊ₓ₎ | products | |
| a + x = 3 | C₃H₃F₅ | CF₃CH₂CHF₂ | 245fa | C₃H₂F₄ | CF₃CH═CHF | 1234ze |
| | | CF₃CF₂CH₃ | 245cb | C₃H₂F₄ | CF₃CF═CH₂ | 1234yf |
| | | CF₃CHFCH₂F | 245eb | C₃H₂F₄ | CF₃CF═H₂ | 1234yf |
| a + x = 4 | C₃H₄F₄ | CF₃CH₂CH₂F | 254fb | C₃H₃F₃ | CF₃CH═CH₂ | 1243zf |

Dehydrofluorination catalysts include aluminum fluoride, fluorided alumina, metals on aluminum fluoride, and metals on fluorided alumina, as disclosed in EP 406748 B1, incorporated herein by reference. Suitable metals include chromium, magnesium (e.g., magnesium fluoride), Group VIIB metals (e.g., manganese), Group IIIB metals (e.g., lanthanum), and zinc. In use, such metals are normally present as halides (e.g., fluorides), as oxides and/or as oxyhalides. Metals on aluminum fluoride and metals on fluorided alumina can be prepared by procedures as described in U.S. Pat. No. 5,731,481 incorporated herein by reference. In one embodiment, when supported metals are used, the total metal content of the catalyst is from about 0.1 to 20 percent by weight, typically from about 0.1 to 10 percent by weight. Preferred catalysts include catalysts consisting essentially of aluminum fluoride and/or fluorided alumina.

Additionally, dehydrofluorination catalysts include oxides, fluorides, and oxyfluorides of magnesium, zinc and mixtures of magnesium and zinc and/or aluminum. A suitable catalyst may be prepared, for example by drying magnesium oxide until essentially all water is removed, e.g., for about 18 hours at about 100° C. The dried material is then transferred to the reactor to be used. The temperature is then gradually increased to about 400° C. while maintaining a flow of nitrogen through the reactor to remove any remaining traces of moisture from the magnesium oxide and the reactor. The temperature is then lowered to about 200° C. and a fluorinating agent, such as HF, or other vaporizable fluorine containing compounds such as SF₄, CCl₃F, CClF₃, CHF₃, CHClF₂, CF₃CH₂F, CF₃CHF₂ and the like, optionally diluted with an inert gas such as nitrogen, is passed through the reactor. The inert gas or nitrogen can be gradually reduced until only HF or other vaporizable fluorine containing compounds is being passed through the reactor. At this point, the temperature can be increased to about 450° C. and held at that temperature to convert the magnesium oxide to a fluoride content corresponding to at least 40 percent by weight, e.g., for 15 to 300 minutes, depending on the fluoriding agent flow rate and the catalyst volume. The fluorides are in the form of magnesium fluoride or magnesium oxyfluoride; the remainder of the catalyst is magnesium oxide. It is understood in the art that fluoriding conditions such as time and temperature can be adjusted to provide higher than 40 percent by weight fluoride-containing material.

Additionally, the dehydrofluorination catalysts could include chromium oxides, fluorided chromium oxides, and cubic chromium trifluoride. Cubic chromium trifluoride may be prepared from CrF₃XH₂O, where X is 3 to 9, preferably 4, by heating in air or an inert atmosphere (e.g., nitrogen or argon) at a temperature of about 350° C. to about 400° C. for 3 to 12 hours, preferably 3 to 6 hours.

Cubic chromium trifluoride is useful by itself, or together with other chromium compounds, as a dehydrofluorination catalyst. Preparation of cubic chromium trifluoride is described in U.S. Pat. No. 6,031,141, incorporated herein by reference. Of note are catalyst compositions comprising chromium wherein at least 10 weight percent of the chromium is in the form of cubic chromium trifluoride, particularly catalyst compositions wherein at least 25 percent of the chromium is in the form of cubic chromium trifluoride, and especially catalyst compositions wherein at least 60 percent of the chromium is in the form of cubic chromium trifluoride. The chromium, including the cubic chromium trifluoride can be supported on and/or physically mixed with materials such as carbon, aluminum fluoride, fluorided alumina, lanthanum fluoride, magnesium fluoride, calcium fluoride, zinc fluoride and the like. Preferred are combinations including cubic chromium trifluoride in combination with magnesium fluoride and/or zinc fluoride.

Additionally, dehydrofluorination catalysts include activated carbon, or three dimensional matrix carbonaceous materials as disclosed in U.S. Pat. No. 6,369,284, incorporated herein by reference; or carbon or metals such as sodium, potassium, rubidium, cesium, yttrium, lanthanum, cerium, praseodymium, neodymium, samarium, chromium, iron, cobalt, rhodium, nickel, copper, zinc, and mixtures thereof, supported on carbon as disclosed in U.S. Pat. No. 5,268,122, incorporated herein by reference. Carbon from any of the following sources is useful for the process of this invention; wood, peat, coal, coconut shells, bones, lignite, petroleum-based residues and sugar. Commercially available carbons which may be used include those sold under the following trademarks: Barneby & Sutcliffe™, Darco™, Nucharm, Columbia JXN™, Columbia LCK™, Calgon PCB, Calgon BPL™, Westvaco™, Norit™, and Barnaby Cheny N B.

Carbon includes acid-washed carbon (e.g., carbon which has been treated with hydrochloric acid or hydrochloric acid followed by hydrofluoric acid). Acid treatment is typically sufficient to provide carbon that contains less than 1000 ppm of ash. Suitable acid treatment of carbon is described in U.S. Pat. No. 5,136,113, incorporated herein by reference. The carbon also includes three dimensional matrix porous carbonaceous materials. Examples are those described in U.S. Pat. No. 4,978,649, incorporated herein by reference. Of note are three dimensional matrix carbonaceous materials which are obtained by introducing gaseous or vaporous carbon-containing compounds (e.g., hydrocarbons) into a mass of granules of a carbonaceous material (e.g., carbon black); decomposing the carbon-containing compounds to deposit carbon on the surface of the granules; and treating the resulting material with an activator gas comprising steam to provide a porous carbonaceous material. A carbon-carbon composite material is thus formed.

The catalytic dehydrofluorination may be suitably conducted at a temperature in the range of from about 200° C. to about 500° C., and, in another embodiment, from about 300° C. to about 450° C. The contact time is typically from about 1 to about 450 seconds, and, in another embodiment, from about 10 to about 120 seconds.

The reaction pressure can be subatmospheric, atmospheric or superatmostpheric. Generally, near atmospheric pressures are preferred. However, the dehydrofluorination can be beneficially run under reduced pressure (i.e., pressures less than one atmosphere).

The catalytic dehydrofluorination can optionally be carried out in the presence of an inert gas such as nitrogen, helium, or argon. The addition of an inert gas can be used to increase the extent of dehydrofluorination. Of note are processes where the mole ratio of inert gas to hydrofluorocarbon undergoing dehydrofluorination is from about 5:1 to about 1:1. Nitrogen is the preferred inert gas.

In the preferred embodiment of dehydrofluorination, the dehydrofluorination of hydrofluorocarbon can be carried out in a reaction zone at an elevated temperature in the presence of a catalyst as described below in the description for the pyrolysis of CF₃CH₂CF₃ to CF₂═CHCF₃ and HF. Appropriate temperatures may be between about 350° C. and about 900° C., and preferably between about 450° C. and about 900° C. The residence time of gases in the reaction zone is typically from about 0.5 to about 60 seconds, and preferably from about 2 seconds to about 20 seconds. The catalytic dehydrofluorination can be carried out in the absence or presence of low levels of an oxidizer such as oxygen or an oxygen containing agent such as air or carbon dioxide. A low level of chlorine gas can also be used. If the process is carried out in the absence of the co-feed of an oxidizer, the dehydrofluorinating catalyst may become deactivated after usage for several hours or days. If this occurs, the process must be shut down and catalyst subjected to a regeneration cycle. The regeneration temperature is ideally between 300-400° C. A stream of air is gradually introduced into the reactor in such a way as not to exceed 375°-400° C. In case of run away reaction, process air feed must be stopped, until the reactor temperature is restored to 375°-400° C. Ideal contact time is between 1-100 seconds. The regeneration cycle is continuously monitored for CO₂ evolution and is concluded when no more CO₂ evolution is observed. The CO₂ evolution is most conveniently monitored by using on line GC. Alternatively, the dehydrofluorination process can be carried out using oxygen containing gas such as air or CO₂. An ideal air feed is between 0.1-1% calculated as oxygen. If an active air gas is cofeed, the catalyst may be run for extended period of time without the need for regular shut down.

Alternatively, the second step of the present invention can comprise the manufacture of a hydrofluoropropene by dehydrofluorination of a hydrofluoropropane in a reaction zone at elevated temperature in the absence of catalyst. For example, producing CF₃CF═CHF by pyrolysis of CF₃CHFCHF₂. The process may be written as: CF₃CHFCHF₂+Δ→CF₃CF═CHF+HF where Δ represents heat and HF is hydrogen fluoride. Pyrolysis, as the term is used herein, means chemical change produced by heating in the absence of catalyst. Pyrolysis reactors generally comprise three zones: a) a preheat zone, in which reactants are brought close to the reaction temperature; b) a reaction zone, in which reactants reach reaction temperature and are at least partially pyrolyzed, and products and any byproducts form; c) a quench zone, in which the stream exiting the reaction zone is cooled to stop the pyrolysis reaction. Laboratory-scale reactors have a reaction zone, but the preheating and quenching zones may be omitted.

The reaction pressure for the dehydrofluorination reaction at elevated temperature in the absence of catalyst may be subatmospheric, atmospheric, or superatmospheric. Generally, near atmospheric pressures are preferred. However, the dehydrofluorination can be beneficially run under reduced pressure (i.e., pressures less than one atmosphere).

The dehydrofluorination at an elevated temperature in the absence of a catalyst may optionally be carried out in the presence of an inert gas such as nitrogen, helium or argon. The addition of an inert gas can be used to increase the extent of dehydrofluorination. Of note are processes where the mole ratio of inert gas to the hydrofluorocarbon undergoing dehydrofluorination is from about 5:1 to about 1:1. Nitrogen is the preferred inert gas.

The reaction zone for either catalyzed or non-catalyzed dehydrofluorination may be a reaction vessel fabricated from nickel, iron, titanium or their alloys, as described in U.S. Pat. No. 6,540,933, incorporated herein by reference. A reaction vessel of these materials (e.g., a metal tube) optionally packed with the metal in suitable form may also be used. When reference is made to alloys, it is meant a nickel alloy containing from about 1 to about 99.9 weight percent nickel, an iron alloy containing about 0.2 to about 99.8 weight percent iron, and a titanium alloy containing about 72 to about 99.8 weight percent titanium. Of note is the use of an empty (unpacked) reaction vessel made of nickel or alloys of nickel such as those containing about 40 weight percent to about 80 weight percent nickel, e.g., Inconel™ 600 nickel alloy, Hastelloy™ C617 nickel alloy or Hastelloy™ C276 nickel alloy.

The hydrofluoroolefins of the present invention may exist as different isomers or stereoisomers. Included are all single configurational isomers, single stereoisomers or any combination thereof. For instance, HFC-1234ze (CF₃CH═CHF) is meant to represent the E-isomer, Z-isomer, or any combination or mixture of both isomers in any ratio.

In the noncatalytic pyrolysis of hydrofluropropane to hydrfluoropropene, the reactor may be of any shape consistent with the process but is preferably a cylindrical tube, either straight or coiled. Although not critical, such reactors typically have an inner diameter of from about 1.3 to about 5.1 cm (about 0.5 to about 2 inches) and a length from 5 to about 8 cm (about 6 to 20 inches). Heat is applied to the outside of the tube, the chemical reaction taking place on the inside of the tube. The reactor and its associated feed lines, effluent lines and associated units should be constructed, at least as regards the surfaces exposed to the reaction reactants and products, of materials resistant to hydrogen fluoride. Typical materials of construction, well-known to the fluorination art, include stainless steels, in particular of the austenitic type, the well-known high nickel alloys, such as Monel® nickel-copper alloys, Hastelloy-based alloys and Inconel® nickel-chromium alloys and copper clad steel. Where the reactor is exposed to high temperature the reactor may be constructed of more than one material. For example, the outer surface layer of the reactor should be chosen for ability to maintain structural integrity and resist corrosion at the pyrolysis temperature, the inner surface layer of the reactor should be chosen of materials resistant to attack by, that is, inert to, the reactant and products. In the case of the present process, the product hydrogen fluoride is corrosive to certain materials. In other words, the reactor may be constructed of an outer material chosen for physical strength at high temperature and an inner material chosen for resistance to corrosion by the reactants and products under the temperature of the pyrolysis.

For the second step of the process of the present invention, it is preferred that the reactor inner surface layer be made of high nickel alloy, that is an alloy containing at least about 50 wt % nickel, preferably a nickel alloy having at least about 75 wt % nickel, more preferably a nickel alloy having less than about 8 wt % chromium, still more preferably a nickel alloy having at least about 98 wt % nickel, and most preferably substantially pure nickel, such as the commercial grade known as Nickel 200. More preferable than nickel or its alloys, the material for the inner surface layer of the reactor is gold. The thickness of the inner surface layer does not substantially affect the pyrolysis and is not critical so long as the integrity of the inner surface layer is intact. The thickness of the inner surface layer is typically from about 10 to about 100 mils (0.25 to 2.5 mm). The thickness of the inner surface layer can be determined by the method of fabrication, the cost of materials, and the desired reactor life.

The reactor outer surface layer is resistant to oxidation or other corrosion and maintains sufficient strength at the reaction temperatures to keep the reaction vessel from failing of distorting. This layer is preferably Inconel® alloy, more preferably Inconel® 600.

Reactors useful for carrying out the present process are tubes comprising the aforementioned materials of construction. Reactors include those wherein the flow of gases through the reactor is partially obstructed to cause back-mixing, i.e. turbulence, and thereby promote mixing of gases and good heat transfer. This partial obstruction can be conveniently obtained by placing packing within the interior of the reactor, filling its cross-section or by using perforated baffles. The reactor packing can be particulate or fibrillar, preferably in cartridge disposition for ease of insertion and removal, has an open structure like that of Raschig Rings or other packings with a high free volume, to avoid the accumulation of coke and to minimize pressure drop, and permits the free flow of gas. Preferably the exterior surface of such reactor packing comprises materials identical to those of the reactor inner surface layer, materials that do not catalyze dehydrofluorination of hydrofluorocarbons and are resistant to hydrogen fluoride. The free volume is the volume of the reaction zone minus the volume of the material that makes up the reactor packing. The free volume is at least about 80%, preferably at least about 90%, and more preferably about 95%.

The pyrolysis which accomplishes the conversion of CF₃CH₂CF₃ to CF₂═CHCF₃ is suitably conducted at a temperature of at least about 700° C., preferably at least about 750° C., and more preferably at least about 800° C. The maximum temperature is no greater than about 1,000° C., preferably no greater than about 950° C., and more preferably no greater than about 900° C. The pyrolysis temperature is the temperature of the gases inside at about the mid-point of the reaction zone.

The residence time of gases in the reaction zone is typically from about 0.5 to about 60 seconds, more preferably from about 2 seconds to about 20 seconds at temperatures of from about 700° to about 900° C. and atmospheric pressure. Residence time is determined from the net volume of the reaction zone and the volumetric feed rate of the gaseous feed to the reactor at a given reaction temperature and pressure, and refers to the average amount of time a volume of gas remains in the reaction zone.

The pyrolysis is preferably carried out to a conversion of the CF₃CH₂CF₃ at least about 25%, more preferably to at least about 35%, and most preferably to at least about 45%. By conversion is meant the portion of the reactant that is consumed during a single pass through the reactor. Pyrolysis is preferably carried out to a yield of CF₃CH═CF₂ of at least about 50%, more preferably at least about 60%, and most preferably at least about 75%. By yield is meant the moles of CF₃CH═CF₂ produced per mole of CF₃CH₂CF₂ consumed.

The reaction is preferably conducted at subatmospheric, or atmospheric total pressure. That is, the reactants plus other ingredients are at subatmospheric pressure or atmospheric pressure. If inert gases are present as other ingredients, as discussed below, the sum of the partial pressures of the reactants plus such ingredients is subatmospheric or atmospheric. Near atmospheric total pressure is more preferred. The reaction can be beneficially run under reduced total pressure (i.e., total pressure less than one atmosphere).

The elimination and addition of HF are reversible processes. Therefore, in the second step, the co-produced HF must be separated from the fluorinated propene to minimize the reverse reaction. This separation is preferably accomplished by processes known in the art such as scrubbing, adsorption or membrane separation.

### Example 1

### Liquid Phase Fluorination of 240 cb to 245 cb

CCl₃CCl₂CH₃+HF→CF₃CF₂CH₃

A catalyst would be activated by condensing anhydrous HF gas (120 grams, 6 moles) in one liter autoclave, equipped with a 100 psig back pressure regulator, and a cold condenser maintained at 5° C. and admitting anhydrous SbCl₅ (5 grams, 0.017 moles) to the autoclave under a dry nitrogen atmosphere. The co-product HCl gas would be vented to a water scrubber. Subsequently 120 grams, 0.55 moles of 240 cb would be added to the autoclave and the mixture stirred continuously at 100° C. for 2 hours. The HCl gas co-product would be vented continuously, and the organic product obtained would be transferred with nitrogen flow 20 ccm and collected at 0° C. The product obtained would be expected to be 60 grams of 245 cb (CF₃CF₂CH₃). A 90% selectivity at 100% conversion would be expected.

### Example 2

### Dehydrofluorination of 245 cb to 1234 yf

CF₃CF₂CH₃→CF₃CF═CH₂

245 cb was fed to a fixed bed reactor containing NI 6%-Cr6% catalyst supported on AlF₃. The catalyst was activated with HF gas at high temperature. The reactor was maintained at 350° C. and the contact time was 89 seconds. A 70% conversion with 85% selectivity towards 1234 yf was obtained.

### Example 3

### Catalyzed Liquid Phase Fluorination of 240 db to 245 eb

CCl₃CHClCH₂Cl+HF→CF₃CHFCH₂F

Anhydrous HF gas (120 grams, 6 moles) would be condensed in one liter autoclave, equipped with a 100 psig back pressure regulator and a cold condenser maintained at 5° C. Anhydrous SbCl5 (5 grams, 0.017 moles) would be admitted to the autoclave under a dry nitrogen atmosphere after activating the catalyst. The co-product, HCl gas, would be vented. Subsequently 240 db (120 grams, 0.55 moles) would be added to the autoclave and the mixture was stirred continuously at 100° C. for 2 hours. The HCl gas co-product would be vented continuously, and the organic product obtained would be transferred with nitrogen flow (20 cc/m) and collected at 0° C. The product expected to be obtained would be 60 grams of 240 eb (CF₃CHFCH₂F). A 90% selectivity at 100% conversion would be expected.

### Example 4

### Dehydrofluorination of 245 eb to 1234 yf

CF₃CHFCH₂F→CF₃CF═CH₂

The catalyst used in Example 5 was be used to dehydrofluorinate 245 eb. 245 eb was fed at 20 cc/m at 340° C. to the reactor described in Example 5. After scrubbing HF co-product and drying the desired organic product, 1234 yf was produced. A 70% selectivity at 85% conversion was achieved.

### Example 5

### Liquid Phase Fluorination of 240 fa to 245 fa

CCl₃CH₂CHCl₂+HF→CF₃CH₂CHF₂

Anhydrous HF gas (120 grams, 6 moles) was condensed in one liter autoclave, equipped with a 100 psig back pressure regulator and a cold condenser maintained at 5° C. Anhydrous SbCl₅ (5 grams, 0.017 moles) was admitted to the autoclave under a dry nitrogen atmosphere after activating the catalyst. The co-product HCl gas was vented to a water scrubber. Subsequently 240 fa (120 grams, 0.55 moles) was added to the autoclave and the mixture stirred continuously at 100° C. for 2 hours. HCl gas co-product was vented continuously, and the organic product obtained transferred via a nitrogen flow (20 cc/m) and collected at 0° C. The product obtained comprises 60 grams of 245 fa (CF₃CF₂CH₃). A 90% selectivity at 100% conversion.

### Example 6

### Catalytic Dehydrofluorination of 245 fa to 1234 ze

This process was carried out using fix bed catalyst reactor. Two catalysts were utilized. A summary of catalysts used and processing conditions is provided in Table 1.

TABLE 3

| | | | Contact | | | | | |
|---|---|---|---|---|---|---|---|---|
| | Temp | | Time | % | Product | Product | Product | Product |
| Catalyst | ° C. | % O₂ | seconds | Conversion | 1 | 2 | 3 | 4 |
| Cr2O3 | 400 | 3 | 45 | 96 | 1.7 | 77.8 | 18.5 | 2 |
| Cr/Ni/AlF3 | 400 | 3 | 26 | 95 | 1.6 | 77 | 20.5 | .9 |
| Cr/Ni/AlF3 | 400 | 3 | 35 | 86 | .6 | 80.5 | 18 | .9 |
| Cr/Ni/AlF3 | 400 | 3 | 39 | 88 | 1.9 | 78.5 | 17.7 | 1.9 |

Product 1 is trifluoropropyne
Product 2 is trans-1234ze (CF₃CH═CHF)
Product 3 is cis-1234ze (CF₃CCH═CHF)
Product 4 is other material(s)

### Example 7

### Dehydrofluorination of 245 fa to 1234 ze Using Aqueous KOH Solution

Feeding 245 fa at 10 cc/m into a 3000 ml (2.7 moles) solution of KOH at room temperature resulted in a 26% conversion to trans-1234 ze 74% by weight and cis-1234ze 23.9% by weight.

### Example 8

### Dehydrofluorination of 245 eb to 1234 yf Using Aqueous KOH Solution

Feeding 245 eb into a 20% KOH at 140° C. resulted in a 95% conversion to 1234 yf with a selectivity of 90% after 6 h.

### Example 9

### Addition of Methyl Chloride to Perchloroethylene

CH₃Cl+CCl₂═CCl₂→CCl₃CCl₂CH₃

A mixture of 1 mole of perchloroethylene, 0.01 moles of anhydrous AlCl₃, could be stirred together at 0° C. Subsequently, methyl chloride gas (20 cc/m) would be bubbled, through the solution. The mixture would be gradually warmed to room temperature. The product expected to be obtained would be mostly high molecular weight material and approximately 10% of a product identified as 240 cb (pentachloropropane).

While the present invention has been described with respect to particular embodiments thereof, it is apparent that numerous other forms and modifications of this invention will be obvious to those skilled in the art. The appended claims and this invention generally should be construed to cover all such obvious forms and modifications which are within the true spirit and scope of the present invention.

1. A process for producing a hydrofluoroolefin of the formula C₃H₍ₐ₊ₓ₋₁₎F₇₋₍ₐ₊ₓ₎ where a=0, 1, 2, 3 or 4 and x=0, 1, 2 or 3 and a+x is one or more, comprising the steps of
a) fluorinating a hydrochlorocarbon of the formula C₃Hₐ₊ₓCl₈₋₍ₐ₊ₓ₎ with HF to form a hydrofluoropropane of the formula C₃Hₐ₊ₓF₈₋₍ₐ₊ₓ₎; and
b) dehydrofluorinating said hydrofluoropropane to form a hydrofluoroolefin of the formula C₃H₍ₐ₊ₓ₋₁₎F₇₋₍ₐ₊ₓ₎.

2. The process of claim 1 wherein said fluorinating occurs in the liquid phase in the presence of a fluorinating catalyst.

3. The process of claim 2 wherein the fluorinating catalyst is selected from antimony, titanium, tin or an ionic liquid.

4. The process of claim 2 wherein said catalyst is present in an amount of at least 0.001 moles per mole of hydrochlorocarbon.

5. The process of claim 2 wherein said catalyst is present in an amount of from about 0.5 to 1 mole per mole of hydrochlorocarbon.

6. The process of claim 1 wherein the molar ratio of hydrochlorocarbon to HF is from about 5:1 to 50:1.

7. The process of claim 1 wherein said fluorinating takes place at a temperature of from about 50° to 130° C.

8. The process of claim 1 wherein said fluorinating occurs at a pressure of form about 2 to 40 bar.

9. The process of claim 1 wherein said dehydrofluorinating occurs in the presence of a dehydrofluorinating catalyst.

10. The process of claim 9 wherein said dehydrofluorinating catalyst is supported or unsupported chromium based catalyst.

11. The process of claim 9 wherein said catalyst further comprises a co-catalyst selected from zinc, nickel or manganese.

12. The process of claim 10 wherein said support is selected from fluorinated alumina, fluorinated chromia, fluorinated magnesia or carbon graphite

13. The process of claim 1 wherein said dehydrofluorinating occurs at a temperature of from about 200° to 900° C.

14. The process of claim 1 wherein the contact time of said dehydrofluorinating ranges from about 1 to 450 seconds.

15. The process of claim 1 wherein said dehydrofluorinating occurs at a pressure of from subatmospheric to superaatmospheric.

16. The process of claim 1 wherein said dehydrofluorinating further comprises feeding a gas selected from oxygen, an oxygen containing gas, chlorine or mixtures thereof.

17. The process of claim 1 wherein said dehydrofluorinating is a pyrolysis reaction.

18. The process of claim 17 wherein said dehydrofluorinating further comprise feeding a gas selected from oxygen, an oxygen containing gas, chlorine or mixtures thereof.

19. The process of claim 1 further comprising the steps of:
c) separating said hydrofluoroolefin from unreacted hydrofluoropropane and any partially dehydrofluorinated material, and
d) recycling said unreacted hydrofluoropropane and any partially dehydrofluorinated material to said dehydrofluorinating step b).

20. The process of claim 1 further comprising the step of removing HF from the hydrofluoroolefin as it is produced.

21. The process of claim 20 wherein said removing is accomplished via scrubbing, adsorption or membrane separation.

22. The process of claim 1 wherein said hydrofluoropropane is selected from CF₃CH₂CHF₂ (245 fa), CF₃CF₂CH₃ (245 cb), CF₃CF₂CH₃ (254 fb) or mixtures thereof.

23. The process of claim 1 wherein said hydrofluoroolefin is selected from CF₃CH═CHF (1234 ze), CF₃CF═CH₂ (1234 yf), CF₃CH═CH₂ (1243 zf) or mixtures thereof.

24. The process of claim 1 wherein said hydrochlorocarbon is formed by telomerizing a chlorine containing group of the formula CHₓCl₄₋ₓ with a two carbon group of the formula C₂HₐCl₄₋ₐ to form said hydrochlorocarbon of the formula C₃Hₐ₊ₓCl₈₋₍ₐ₊ₓ₎.
